(19) United States
(12) Patent Application Publication
(10) Pub. No.: US 2018/0140290 A1
(43) Pub. Date: May 24, 2018

(54) Dynamic Retractor System for Laparotomies and Cesarean Sections

(71) Applicant: MedInvent Limited, Romsey (GB)

(72) Inventor: Dharmesh Kapoor, Romsey (GB)

(73) Assignee: MedInvent Limited, Romsey (GB)

(21) Appl. No.: 15/358,659

(22) Filed: Nov. 22, 2016

(51) Int. Cl. A61B 17/02; A61B 90/50

(52) U.S. Cl. CPC A61B 17/0206; A61B 90/50; A61B 17/0218; A61B 17/02

(57) ABSTRACT

A retractor system for use in dynamically retracting the skin adjacent to an incision in a panniculus of a patient during a laparotomy or Cesarean section includes a support frame with at least one support member disposed above the panniculus of the patient, and an upper retractor that includes: a first resilient member including a first support member end attached to the at least one support member, and a first patient attachment end for attaching to the skin of the patient adjacent the incision in the panniculus; and a second resilient member including a second support member end attached to the at least one support member, and a second patient attachment end for attaching to the skin of the patient adjacent the incision in the panniculus. The first and second resilient members are responsive to the respiration of the patient.

### FIELD

The present invention relates to a dynamic retractor system for use in surgical procedures involving incisions into the abdominal cavity of a patient. Such surgical procedures are called laparotomies. A Cesarean section is one type of laparotomy.

### BACKGROUND

A Cesarean section is performed in twenty-five to thirty percent of births in Europe and the United States. Around eight percent of all deliveries, however, are complicated by high body mass index or BMI (i.e., greater than forty) during pregnancy. The incidence of Cesarean sections in this group of morbidly obese women is nearly fifty percent.

A Cesarean section involves several steps. First, the patient's abdomen is painted with a sterile antiseptic solution to cleanse the skin of microorganisms and reduce the risk of infection. A skin incision is then made transversely or vertically in the lower part of the patient's abdomen. The fat layer is dissected and the rectus muscle sheath/fascia is opened using scissors or a knife. Next, the rectus muscles are manually separated, the parietal peritoneum is opened, and a Doyen retractor is inserted below the uterus. The visceral peritoneum is then opened and the Doyen retractor is repositioned to hide the urinary bladder below it. After the uterus has been opened and the baby is ready to be delivered, more space is needed to ensure safe delivery of the baby. To create that space, the Doyen retractor is removed. After delivery, the Doyen retractor is re-inserted. The uterus is then closed with sutures in one to two layers. Before closing the rectus muscle sheath/fascia, the Doyen retractor is once again removed to check for any bleeding points and assure hemostasis. The patient's abdomen is then closed serially in layers (i.e., the fat layer then the skin layer).

During a Cesarean section, the main surgeon usually has one assistant. In some instances, however, extra help is needed to reflect away the fat from the upper part of the abdominal incision (e.g., the part of the abdominal incision nearer to the patient's head if the incision is transverse). Skin in this area is known as the “panniculus,” and oftentimes an extra doctor, nurse, or midwife is needed to manually retract the panniculus so that the surgical site is accessible.

It is common for obese patients to have a panniculus. A patient may also have a panniculus due to loose tissue as a result of a previous pregnancy or due to massive weight loss. FIG. 1 shows a patient 2 with a panniculus 4. The significance or extent of the panniculus of a patient can vary. A grade 1 panniculus covers the hairline of the mons pubis but not the genitalia. A grade 2 panniculus extends to cover the genitalia. A grade 3 panniculus extends to cover the upper thigh. A grade 4 panniculus extends to cover the mid thigh. A grade 5 panniculus extends to cover the knees or beyond. The panniculus shown in FIG. 1 is a grade 3 panniculus. Not retracting and supporting the panniculus properly during abdominal surgery can lead to serious complications.

In a surgical procedure, including laparotomies and Cesarean sections, it is desirable to implement a retraction solution that provides for safe and effective retraction and support of the panniculus, no matter the grade, while not requiring extra personnel in the operating room.

For example, to manage an overhanging panniculus, some surgeons use Montgomery straps applied to the upper abdomen of the patient. The straps are tied to a bed rail to retract the panniculus cephalad (i.e., toward the patient's head). In some cases, the surgeon will use a chain with a Doyen retractor attached to one end to retract this upper abdomen skin. But this technique places the skin under an amount of tension that can immobilize the upper part of the abdomen such that it does not move with the respiration of the patient, often creating breathing problems for the patient and the obese patient in particular.

Another technique for performing a Cesarean section on a morbidly obese patient involves using a plastic sheet with adhesive, such as a Traxi®, to retract the panniculus before making the skin incision. One negative aspect of this solution is that the Traxi® can only be applied to the patient once the skin antiseptic solution has dried, which takes about three to five minutes. This time delay is crucial when an emergency Cesarean section must be performed. Use of the Traxi® also leads to the skin incision being a lot higher (i.e., more cephalic) than it would be otherwise. This technique also results in an amount of tension on the panniculus that can immobilize the abdomen such that it does not move with the respiration of the patient. As such, it places undue pressure on the diaphragm of the mother during the Cesarean section, which can lead to breathing problems. Similar problems are encountered in other abdominal surgical procedures on obese patients. Furthermore, the Traxi® retracts only the skin and not the internal organs, such that upon entering the peritoneal cavity, it is still necessary to use retractors that require an assistant to hold them. Such retractors are typically single-use retractors formed of two rubber-elastic rings connected by a polyurethane sheet to retract the internal organs away from the site of the operation. When using such retractors, one ring is placed intra-abdominally while the other is disposed outside the body of the patient, which allows the interconnecting polyurethane sheet to retract the tissue around the incision site. Examples of such single-use retractors include Alexis O-rings and Mobius rings. These retractors, however, can only be used after making an incision in the skin and entering the abdominal cavity. If a surgeon has to achieve both objectives of retraction (i.e., retraction of the panniculus and retraction of the internal organs) when using this technique, then the surgeon must use a Traxi® or similar device to retract the panniculus and a double-ring retractor to retract the internal organs. Employing both of these devices can cause surgical complications and requires an assistant.

Other retractors used in abdominal surgery are not used during Cesarean sections because they occupy a lot of space in the abdominal cavity and can potentially harm the baby. They also cannot be inserted until the opening of the peritoneal cavity.

When using the foregoing techniques, the retracting member is static (i.e., not dynamic) such that the panniculus is subjected to a higher amount of tension when the patient exhales and the diaphragm of the patient contracts. Conversely, the panniculus is subjected to a lower amount of tension when the patient inhales and the diaphragm of the patient relaxes. In either case, however, the tension levels remain high enough that they can nonetheless immobilize the abdomen such that it does not move with the respiration of the patient. Immobilization of the abdomen can have adverse consequences on the patient, namely increased pressure on the patient's diaphragm and an attendant reduction in the patient's ability to breathe.

A Yasargil hook is another example of a retraction device, but Yasargil hooks are typically used for operations on the skull and brain of a patient. For example, the spring in the Yasargil hook is intended to provide gentle traction to retract a layer of the skull to expose the surgical site. The spring in the Yasargil hook therefore tends to be small (e.g., the diameter is typically less than 5 mm and the length is typically 7 cm). Moreover, such a spring can withstand only a minimal extension before it irreversibly deforms and becomes unsuitable for further medical use. The smaller spring of the Yasargil hook also cannot withstand surgical sterilization at temperatures high enough to ensure the elimination of bacteria and other microorganisms. Consequently, the spring in the Yasargil hook cannot be used effectively for a Cesarean section and other similar abdominal surgical procedures.

As such, there is a need to dynamically retract the panniculus to lower the amount of tension to which the panniculus is subjected to permit movement with the patient's respiration. At the same time, there is also a need to minimize the number of personnel in the operating room during the laparotomy or Cesarean section.

### SUMMARY

In an embodiment, the present invention provides a retractor system for use in dynamically retracting the skin adjacent to an incision in a panniculus of a patient during a laparotomy or Cesarean section, the patient being disposed supine on a surface of an operating table, the operating table including a head end closer to the head of the supine patient than the patient's feet, and a foot end closer to the feet of the supine patient than the patient's head, the foot end being opposite the head end. The retractor system includes a support frame configured to be disposed at the head end of the operating table, the support frame including at least one support member, the at least one support member being configured to be disposed at a support member height above the panniculus of the patient, the support member height being measured in a direction approximately orthogonal to the surface of the operating table. The retractor system also includes an upper retractor that comprises: a first resilient member including a first support member end attached to the at least one support member and a first patient attachment end opposite the first support member end, the first patient attachment end configured to attach to the skin of the patient adjacent the incision in the panniculus; and a second resilient member including a second support member end attached to the at least one support member and a second patient attachment end opposite the second support member end, the second patient attachment end configured to attach to the skin of the patient adjacent the incision in the panniculus. The first and second resilient members are configured to be responsive to the respiration of the patient. In another embodiment of the invention, the retractor system comprises a single resilient member.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings, which illustrate the following:

FIG. 1 is a profile view of a patient with a panniculus disposed supine on a surface of an operating table;

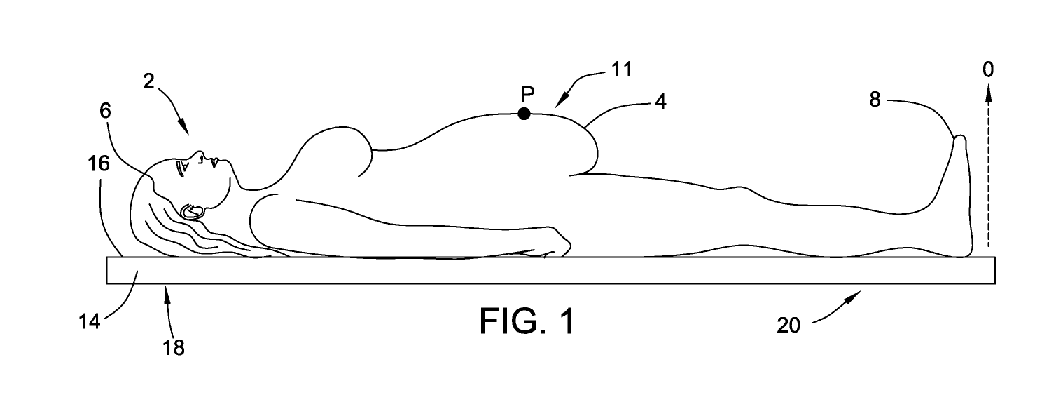

FIG. 2 is a schematic view showing the retractor system described herein in use on the patient shown in FIG. 1, wherein the incision in the patient is a transverse incision;

FIG. 3 is a profile view of the retractor system of FIG. 2 in use on the patient shown in FIG. 1;

FIG. 4 is a schematic view showing the retractor system described herein in use on the patient shown in FIG. 1, wherein the incision in the patient is a vertical incision; and

FIG. 5 is a schematic view of a first lower retractor rod of a lower retractor of the retractor system described herein.

### DETAILED DESCRIPTION

In an embodiment, the present invention provides a dynamic retractor system for use during a laparotomy or Cesarean section. The retractor system enables the main surgeon to perform the laparotomy or Cesarean section while minimizing adverse effects on the patient and the need for an assistant to manually retract skin or vital organs of the patient that could otherwise be susceptible to injury during the operation. In this manner, the retractor system allows retraction of both the skin of the patient and internal retraction.

The retractor system is dynamic in its function, in that it will move with and respond to the respiration of the patient and minimize breathing difficulties often encountered in a laparotomy or Cesarean section, particularly in the obese patient, in part by reducing the pressure on the patient's diaphragm resulting from the weight of the patient's panniculus. The dynamic nature of the retractor system permits retraction of the relevant tissues to expose the operating site while at the same time maximizing the variability in the amount of tension to which the panniculus is subjected during retraction.

To achieve this dynamic function, the retractor system includes one or more resilient members that are responsive to the respiration of the patient. Each resilient member is configured to contract upon the inhalation of the patient and configured to extend upon the exhalation of the patient. In this manner, the retractor system lowers the amount of tension to which the panniculus is subjected during retraction so as to not immobilize the abdomen, correspondingly minimizing the risks to patients associated with static retraction techniques. At the same time, the retractor system also maximizes the variability in the amount of tension that would be absent when using static retraction techniques. The resilient members contemplated herein are strong enough to elevate the panniculus of the patient, yet not impair the dynamic nature of the retraction. The resilient members are resilient in that they are able to recoil or spring back into their original length and shape after being contracted and extended.

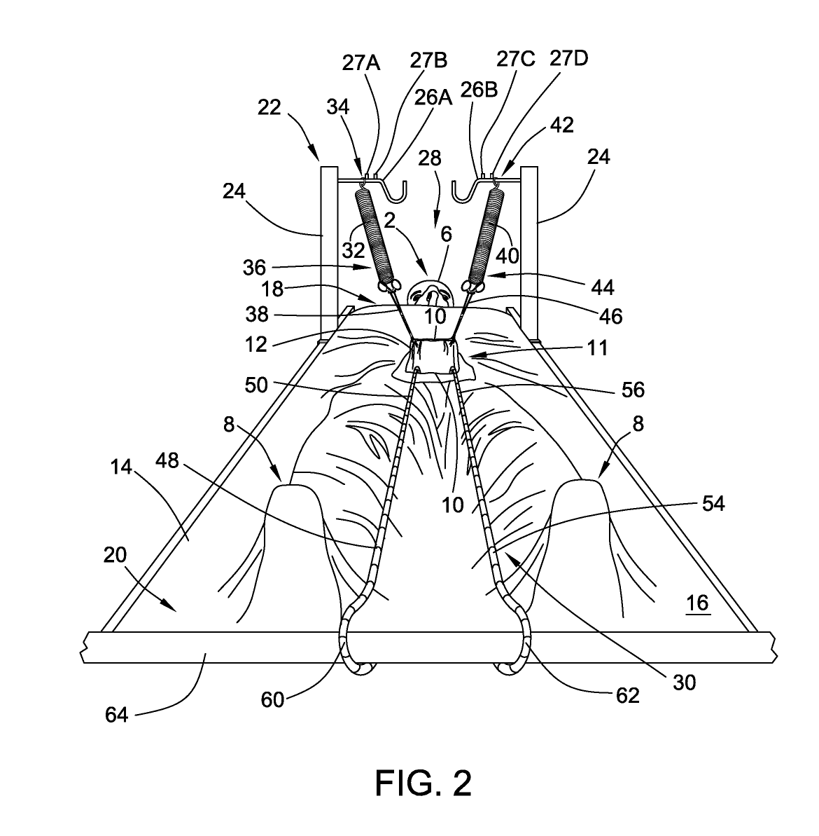

The retractor system is shown schematically in FIGS. 2-5. The retractor system includes a support frame 24, an upper retractor 28, and, optionally, a lower retractor 30. As shown in FIG. 2, a patient 2 is disposed supine (i.e., laying face upward) on a surface 16 of an operating table 14. The operating table 14 includes a head end 18 closer to the head 6 of the supine patient 2 than the patient's feet 8 and a foot end 20 closer to the feet 8 of the supine patient 2 than the patient's head 6. The foot end 20 of the operating table 14 is opposite the head end 18 of the operating table 14.

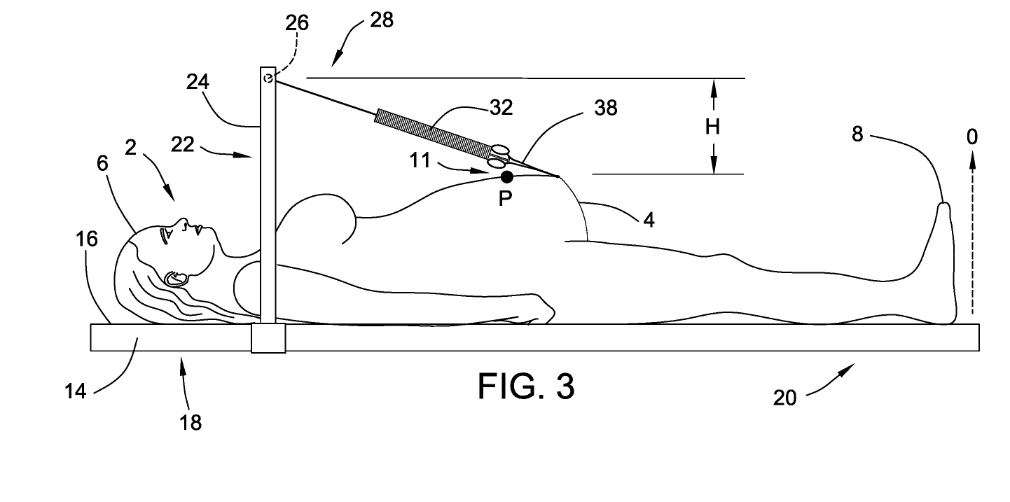

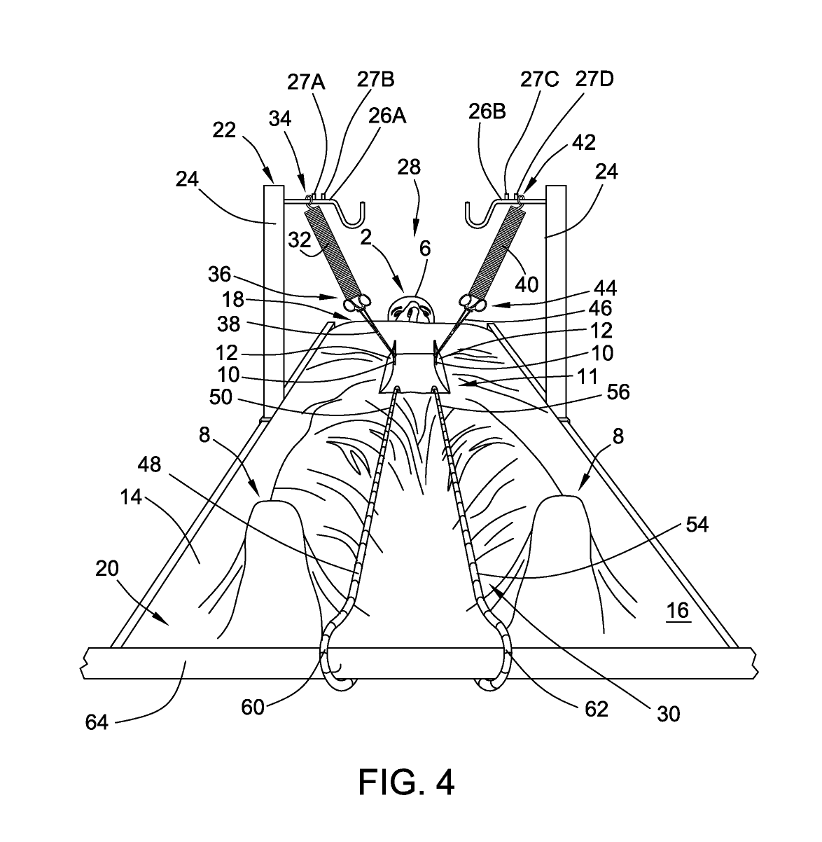

As shown in FIG. 3, one version of the support frame 24 is disposed at the head end 18 of the operating table 14 (i.e., nearer to the patient's head 6 than the patient's feet 8). This version of support frame 24 includes one or more support members 26 (e.g., first support member 26A to one lateral side of the patient 2 and second support member 26B to the other lateral side of the patient 2, as shown in FIG. 2) that are affixed to the support frame 24. The support members 26 are disposed at a support member height H above the panniculus 4 of the patient 2. The support member height H is measured from a point P on the panniculus 4 of the patient 2 that, in a direction O approximately orthogonal to the surface 16 of the operating table 14 on which the patient 2 is disposed, is approximately farthest from the surface 16 of the operating table 14 when the patient 2 is supine. The point P is determined prior to the surgeon making the incision 10 in the patient 2. In one embodiment, for example, the support member height H may range from 0.2 m to 1 m. It is contemplated that the support member height H could be increased or decreased after the surgeon makes an incision 10 (FIGS. 2, 4) in patient 2.

The position of the support frame 24 along the length of the operating table 14 can vary. For example, in the case of a transverse incision 10 (as shown in FIGS. 2-3), the support frame 24 is positioned closer to the head 6 of the patient 2 than to the site of the incision 10 (e.g., the support frame 24 is positioned along the operating table 14 at approximately the shoulder of the supine patient 2). In this manner, the panniculus 4 is pulled not only upward, but also in the direction of the head 6 of the patient 2. In other circumstances it may be desirable to move the support frame 24 closer to the site of the incision 10. For example, in the case of a vertical incision 10 (as shown in FIG. 4), the support frame 24 is positioned closer to the site of the incision 10 than to the head 6 of the patient 2 (e.g., the support frame 24 is positioned along the operating table 14 approximately in line with the incision 10). In this manner, the panniculus 4 is pulled not only upward, but also to the lateral sides of the patient 2. In any case, however, the support frame 24 is still disposed at the head end 18 of the operating table 14.

To retain the first resilient member 32 and the second resilient member 40 on the support members 26, the support members 26 may include a number of control stops 27. For example, first support member 26A may include first control stop 27A and second control stop 27B, while second support member 26B may include third control stop 27C and fourth control stop 27D. In this manner, the first member support end 34 of the first resilient member 32 can be retained on the first support member 26A by either first control stop 27A or second control stop 27B so that the first member support end 34 does not slide along the first support member 26A during retraction. Similarly, the second member support end 42 of the second resilient member 40 can be retained on the second support member 26B by either third control stop 27C or fourth control stop 27D so that the second member support end 42 does not slide along the second support member 26B during retraction. For example, with the configuration shown in FIG. 2, the extent of the retraction of the panniculus 4 by first resilient member 32 can be increased by moving the first support member end 34 of first resilient member 32 outward from adjacent second control stop 27B to adjacent first control stop 27A. It is also contemplated that the positions of the control stops 27 along the support members 26 may be adjustable.

A preferred upper retractor 28 is shown in FIGS. 2-3. It includes a first resilient member 32 with a first support member end 34 at one end for attaching the first resilient member 32 to a support member 26 on the support frame 24. At the opposing end of the first resilient member 32 is a first patient attachment end 36 for attaching the first resilient member 32 to the patient 2, in particular to the skin 12 of the patient 2 adjacent the incision 10 in the panniculus 4. The preferred upper retractor 28 also includes a second resilient member 40 with a second support member end 42 at one end for attaching the second resilient member 40 to a support member 26 on the support frame 24. At the opposing end of the second resilient member 40 is a second patient attachment end 44 for attaching the second resilient member 40 to patient 2, in particular to the skin 12 of patient 2 adjacent the incision 10 in the panniculus 4.

The support frame 24 can be fixed in place at the head end 18 of the operating table 14. For example, the support frame 24 may comprise one or more IV stands or IV poles configured to be mounted to the operating table 14, as shown in FIG. 3. In this manner, the support frame 24 is able to remain in place even while subjected to the torque generated by the weight of the panniculus 4 attached to the first resilient member 32 and/or second resilient member 40. Other versions of support frame 24 may be constructed, for example as a single component in the shape of an upside-down U, to receive one or more retractors.

The first patient attachment end 36 and second patient attachment end 44 may comprise, respectively, a first clamp 38 and a second clamp 46. First clamp 38 and second clamp 46 are atraumatic in nature, in that they prevent necrosis of the skin 12 (e.g., the rectus sheath/fascia) to which they are attached. Alternatively, a single clamp with appropriately designed atraumatic jaws may be employed, to be attached along a wider section of the sectioned panniculus, for example.

The first resilient member 32 and second resilient member 40 may be employed simultaneously. For example, when the incision 10 in the abdomen 11 of the patient 2 is transverse (i.e., running from the left side to right side or right side to left side of the patient 2, as shown in FIG. 2), the first resilient member 32 and second resilient member 40 may both be attached on the same side of the incision 10, namely the side of the incision 10 closer to the head 6 of the patient 2. When the incision 10 in the abdomen 11 of the patient 2 is vertical (i.e., running in a direction from the feet 8 to the head 6 or from the head 6 to the feet 8 of the patient 2, as shown in FIG. 4), the first resilient member 32 may be attached on the left or right side of the incision 10, while the second resilient member 40 may be attached on the opposing side of the incision 10. It is also contemplated that only one of the first resilient member 32 and the second resilient member 40 may be used during a laparotomy or Cesarean section. For example, when the panniculus 4 of the patient 2 is less significant, whether due to the grade of the panniculus 4 or the body mass index of the patient 2, it may only be necessary to use the first resilient member 32.

The first resilient member 32 and second resilient member 40 are responsive to the respiration of the patient 2. For example, during inhalation, the diaphragm of the patient 2 relaxes and the lungs of the patient 2 expand. Consequently, the panniculus 4 of the patient 2 tends to be pushed outward, such that the first resilient member 32 and second resilient member 40 contract. During exhalation, the diaphragm of the patient 2 contracts and the lungs of the patient 2 shrink. Consequently, the panniculus 4 of the patient 2 tends to be drawn inward, such that the first resilient member 32 and second resilient member 40 extend. In this manner the first resilient member 32 and second resilient member 40 minimize the amount of tension to which the panniculus 4 is subjected during the respiration of the patient 2 while still permitting retraction of the relevant tissues to expose the operating site.

First resilient member 32 and second resilient member 40 can each comprise a customizable spring that can elevate the panniculus 4 of a patient 2 during a laparotomy or Cesarean section. The spring rates (i.e., the amount of force that is needed to compress the springs by a particular distance) of the first resilient member 32 and second resilient member 40 can be customized to the body mass index of the patient 2 and/or to the grade and mass of the panniculus 4 of the patient 2. In this manner, the first resilient member 32 and second resilient member 40 provide the ability to dynamically retract the panniculus 4, in that the first resilient member 32 and second resilient member 40 move with the respiration of the patient 2 during the retraction. This configuration reduces the breathing difficulties that occur with prior art techniques that involve static retraction.

For example, in an embodiment, the spring rates of the first resilient member 32 and second resilient member 40 can be in the range of 0.25 N/mm to 1.5 N/mm.

The first resilient member 32 and second resilient member 40 can have the same spring rate. Alternatively, the first resilient member 32 and second resilient member 40 can have different spring rates. For example, the first resilient member 32 and second resilient member 40 may have different spring rates when the incision 10 on the patient 2 is vertical, and the extent of the panniculus 4 to one side of the incision 10 is more significant than the extent of the panniculus 4 to the other side of the incision 10. It may also be desirable to have different spring rates when the incision into the panniculus is non-symmetrical.

The ability of the first resilient member 32 and second resilient member 40 to extend under load and return to their original length and shape once the laparotomy or Cesarean section is over surpasses that of springs used in other retraction devices (e.g., the Yasargil hook). The first resilient member 32 and second resilient member 40 may also be constructed without pre-loading (i.e., the first resilient member 32 and the second resilient member 40 are not in tension when not under load) and to have spaces between their coils to prevent microorganisms from nesting therein to enable surgical cleaning and sterilization at high temperatures. In an embodiment, the first resilient member 32 and the second resilient member 40 may be formed from steel wires with diameters ranging from 3 mm to 5 mm and the diameters of the coils of the first resilient member 32 and the second resilient member 40 may be on the order of 30 mm to 40 mm, which is significantly larger than a spring used in a Yasargil hook. In an embodiment, the lengths of the first resilient member 32 and second resilient member 40 are on the order of 50 cm.

The first resilient member 32 and second resilient member 40 can take a form other than that of a customizable spring. For example, the first resilient member 32 and second resilient member 40 can each comprise a hydraulic device or a pneumatic device that permits the same functionality as a customizable spring. The hydraulic and pneumatic devices may be adjustable so as to permit the surgeon to vary the desired amount of tension. Similarly, the first resilient member 32 and second resilient member 40 can each comprise an elastic band (e.g., a band made from rubber). No matter their form, however, the first resilient member 32 and second resilient member 40 permit retraction of the relevant tissues to expose the operating site while at the same time minimizing the amount of tension to which the panniculus 4 is subjected while it is retracted.

The lower retractor 30 of the retractor system 22 is shown in FIGS. 2 and 4-5. The lower retractor 30 of the retractor system 22 replaces the Doyen retractor that is typically held by an assistant during a laparotomy or Cesarean section.

As shown in FIG. 2, the lower retractor 30 comprises a first lower retractor rod 48 and a second lower retractor rod 54. First lower retractor rod 48 and a second lower retractor rod 54 have shape-retaining properties in that they assume the shape into which they are bent and retain this shape (i.e., the first lower retractor rod 48 and the second lower retractor rod 54 are plastically deformable). As such, an assistant is not needed to hold the first lower retractor rod 48 or the second lower retractor rod 54 during a laparotomy or Cesarean section. By extension, an assistant is also not needed to remove the first lower retractor rod 48 or the second lower retractor rod 54 during a baby's delivery in a Cesarean section.

The first lower retractor rod 48 includes a third patient attachment end 50 configured to attach to the skin 12 of the patient 2 adjacent the incision 10 in the panniculus 4. The second lower retractor rod 54 includes a fourth patient attachment end 56 configured to attach to the skin 12 of the patient 2 adjacent the incision 10 in the panniculus 4. For example, the third patient attachment end 50 and the fourth patient attachment end 56 may be attached to the visceral bladder peritoneum of the patient 2.

The first lower retractor rod 48 includes a first support end 60 opposite the third patient attachment end 50. The second lower retractor rod 54 includes a second support end 62 opposite the fourth patient attachment end 56. The first support end 60 and the second support end 62 are configured to be attached to a rail 64 disposed at the foot end 20 of the operating table 14.

The second lower retractor rod 54, at least in part, can be approximately parallel to the first lower retractor rod 48 when the first lower retractor rod 48 and the second lower retractor rod 54 are employed in a laparotomy or Cesarean section utilizing a vertical incision 10.

The first lower retractor rod 48 and the second lower retractor rod 54 may employed simultaneously. For example, when the incision 10 in the abdomen 11 of the patient 2 is transverse, the first lower retractor rod 48 and the second lower retractor rod 54 may both be attached on the same side of the incision 10, namely the side of the incision 10 closer to the feet 8 of the patient 2. When the incision 10 in the abdomen 11 of the patient 2 is vertical, the first lower retractor rod 48 may be attached on the left or right side of the incision 10, while the second lower retractor rod 54 may be attached on the opposing side of the incision 10. It is also contemplated that only one of the first lower retractor rod 48 and the second lower retractor rod 54 may be used during a laparotomy or Cesarean section. For example, when the panniculus 4 of the patient 2 is less significant, whether due to the grade of the panniculus 4 or the body mass index of the patient 2, it may only be necessary to use the first lower retractor rod 48.

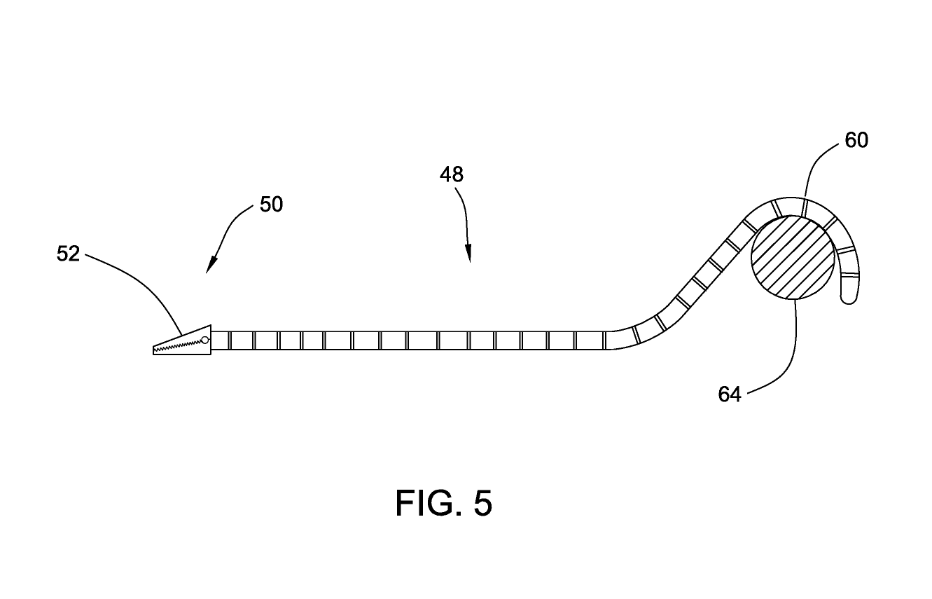

As shown in more detail in FIG. 5, the third patient attachment end 50 of the first lower retractor rod 48 may comprise a third clamp 52. Similarly, the fourth patient attachment end 56 of the second lower retractor rod 54 may comprise a fourth clamp (not shown). Given the similarities between the first lower retractor rod 48 and the second lower retractor rod 54 and their components, however, only the first lower retractor rod 48 is shown in FIG. 5. Like the first clamp 38 and the second clamp 46, the third clamp 52 and fourth clamp 58 are both atraumatic in nature. The clamp configuration may take different shapes—they may be wider, for example, when only a single lower retractor rod is employed.

The first lower retractor rod 48 and the second lower retractor rod 54 can be made of a plastically deformable material such as gooseneck tubing or LocLine®. Alternatively, the first lower retractor rod 48 and the second lower retractor rod 54 may comprise resilient members (e.g., springs), similar to the first resilient member 32 and the second resilient member 40, to protect the surgical site from being exposed to excessive tension during retraction of the skin 12 adjacent the incision 10. In either case, the first lower retractor rod 48 and the second lower retractor rod 54 can be metallic or synthetic, and can also be manufactured in varying sizes, diameters, and rigidities.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE NUMERALS

2 patient
4 panniculus
6 head
8 feet
10 incision
11 abdomen
12 skin
14 operating table
16 surface
18 head end
20 foot end
22 retractor system
24 support frame
26 support member
26A first support member
26B second support member
27 control stop
27A first control stop
27B second control stop
27C third control stop
27D fourth control stop
28 upper retractor
30 lower retractor
32 first resilient member
34 first support member end
36 first patient attachment end
38 first clamp
40 second resilient member
42 second support member end
44 second patient attachment end
46 second clamp
48 first lower retractor rod
50 third patient attachment end
52 third clamp
54 second lower retractor rod
56 fourth patient attachment end
60 first support end
62 second support end
64 rail
H support member height
O direction approximately orthogonal to surface of operating table
P point

What is claimed is:

1. A retractor system for use in dynamically retracting the skin adjacent to an incision in a panniculus of a patient during a laparotomy or Cesarean section, the patient being disposed supine on a surface of an operating table, the operating table including a head end closer to the head of the supine patient than the patient's feet, and a foot end closer to the feet of the supine patient than the patient's head, the foot end being opposite the head end, the retractor system comprising:
a support frame configured to be disposed at the head end of the operating table, the support frame including at least one support member, the at least one support member being configured to be disposed at a support member height above the panniculus of the patient, the support member height being measured in a direction approximately orthogonal to the surface of the operating table; and
an upper retractor comprising:
a first resilient member including a first support member end attached to the at least one support member and a first patient attachment end opposite the first support member end, the first patient attachment end configured to attach to the skin of the patient adjacent the incision in the panniculus; and
a second resilient member including a second support member end attached to the at least one support member and a second patient attachment end opposite the second support member end, the second patient attachment end configured to attach to the skin of the patient adjacent the incision in the panniculus,
wherein the first resilient member and second resilient member are configured to be responsive to the respiration of the patient.

2. The retractor system of claim 1, wherein the first resilient member has a first spring rate and the second resilient member has a second spring rate, and
wherein the first spring rate and the second spring rate are determined based on a body mass index of the patient.

3. The retractor system of claim 2, wherein the first spring rate and the second spring rate are determined based on a grade of the panniculus.

4. The retractor system of claim 1, wherein the first resilient member has a first spring rate and the second resilient member has a second spring rate, and
wherein the first spring rate and the second spring rate are determined based on a grade of the panniculus.

5. The retractor system of claim 1, wherein the respiration of the patient includes inhalation and exhalation, and the first resilient member and the second resilient member are configured to contract upon the inhalation of the patient and configured to extend upon the exhalation of the patient.

6. The retractor system of claim 1, wherein the first patient attachment end comprises a first clamp and the second patient attachment end comprises a second clamp.

7. The retractor system of claim 1, wherein the at least one support member comprises a first support member disposed on one lateral side of the supine patient and a second support member disposed on an opposite lateral side of the patient.

8. The retractor system of claim 7, wherein the first support member end of the first resilient member is attached to the first support member and the second support member end of the second resilient member is attached to the second support member.

9. The retractor system of claim 1, further comprising a lower retractor, the lower retractor comprising:
a first lower retractor rod including a third patient attachment end configured to attach to the skin of the patient adjacent the incision in the panniculus; and
a second lower retractor rod including a fourth patient attachment end configured to attach to the skin of the patient adjacent the incision in the panniculus,
wherein the first lower retractor rod and the second lower retractor rod are plastically deformable.

10. The retractor system of claim 9, wherein the third patient attachment end comprises a third clamp and the fourth patient attachment end comprises a fourth clamp.

11. The retractor system of claim 9, wherein the first lower retractor rod includes a first support end opposite the third patient attachment end, the second lower retractor rod includes a second support end opposite the fourth patient attachment end, and the first support end and the second support end are configured to be attached to a rail disposed at the foot end of the operating table.

12. A retractor system for use in dynamically retracting the skin adjacent to an incision in a panniculus of a patient during a laparotomy or Cesarean section, the patient being disposed supine on a surface of an operating table, the operating table including a head end closer to the head of the supine patient than the patient's feet, and a foot end closer to the feet of the supine patient than the patient's head, the foot end being opposite the head end, the retractor system comprising:
a support frame configured to be disposed at the head end of the operating table, the support frame including at least one support member, the at least one support member being configured to be disposed at a support member height above the panniculus of the patient, the support member height being measured in a direction approximately orthogonal to the surface of the operating table; and
an upper retractor comprising:
a resilient member including a support member end attached to the at least one support member and a patient attachment end opposite the first support member end, the patient attachment end configured to attach to the skin of the patient adjacent the incision in the panniculus,
wherein the resilient member is configured to be responsive to the respiration of the patient.

13. A method for dynamically retracting the skin adjacent to an incision in a panniculus of a patient during a laparotomy or Cesarean section, the patient being disposed supine on a surface of an operating table, the operating table including a head end closer to the head of the supine patient than the patient's feet, and a foot end closer to the feet of the supine patient than the patient's head, the foot end being opposite the head end, the method comprising:
disposing a support frame at the head end of the operating table, the support frame including at least one support member, the at least one support member, upon the disposing of the support frame at the head end of the operating table, being at a support member height above the panniculus of the patient, the support member height being measured in a direction approximately orthogonal to the surface of the operating table;
attaching a first support member end of a first resilient member to the at least one support member;
attaching a second support member end of a second resilient member to the at least one support member;
attaching a first patient attachment end of the first resilient member to the skin of the patient adjacent the incision in the panniculus, the first patient attachment end being opposite the first support member end; and
attaching a second patient attachment end of the second resilient member to the skin of the patient adjacent the incision in the panniculus, the second patient attachment end being opposite the second support member end,
wherein the first resilient member and second resilient member are configured to be responsive to the respiration of the patient.

14. The method of claim 12, wherein the first resilient member has a first spring rate and the second resilient member has a second spring rate, and
wherein the first spring rate and the second spring rate are determined based on a body mass index of the patient.

15. The method of claim 13, wherein the first spring rate and the second spring rate are determined based on a grade of the panniculus.

16. The method of claim 12, wherein the first resilient member has a first spring rate and the second resilient member has a second spring rate, and
wherein the first spring rate and the second spring rate are determined based on a grade of the panniculus.

17. The method of claim 12, wherein the respiration of the patient includes inhalation and exhalation, and the first resilient member and the second resilient member are configured to contract upon the inhalation of the patient and configured to extend upon the exhalation of the patient.

18. The method of claim 12, wherein the first patient attachment end comprises a first clamp and the second patient attachment end comprises a second clamp.

19. The method of claim 12, wherein the at least one support member comprises a first support member disposed on one lateral side of the supine patient and a second support member disposed on an opposite lateral side of the patient.

20. The method of claim 18, wherein the first support member end of the first resilient member is attached to the first support member, and the second support member end of the second resilient member is attached to the second support member.
